(19) United States
(12) Patent Application Publication
(10) Pub. No.: US 2003/0212088 A1
(43) Pub. Date: Nov. 13, 2003

(54) Methods and compositions for treating, preventing or delaying onset of a neoplasm

(76) Inventor: Keith Friend, Flemington, NJ (US)

Correspondence Address: DINSMORE & SHOHL, LLP, 1900 CHEMED CENTER, 255 EAST FIFTH STREET, CINCINNATI, OH, 45202, US

(21) Appl. No.: 10/423,761

(22) Filed: Apr. 25, 2003

Related U.S. Application Data
(60) Provisional application No. 60/375,890, filed on Apr. 26, 2002.

(51) Int. Cl. A61K 31/4745

(52) U.S. Cl. 514/283

(57) ABSTRACT

Methods of treating, preventing or delaying onset of a neoplasm comprise administering to an individual an anti-neoplastic agent and a growth hormone receptor antagonist. Methods for reducing tumor weight or volume comprise administering to an individual an anti-neoplastic agent and a growth hormone receptor antagonist. Compositions comprise an anti-neoplastic agent and a growth hormone receptor antagonist.

### RELATED APPLICATION

The present application claims priority under 35 U.S.C. §119 of U.S. Provisional Application Serial No. 60/375,890 filed Apr. 26, 2002.

### FIELD OF THE INVENTION

The present invention is directed to methods and compositions for treating, preventing and/or delaying onset of a neoplasm. The methods comprise administering to an individual an anti-neoplastic agent and a growth hormone receptor antagonist.

### BACKGROUND OF THE INVENTION

Many individuals either have a benign or malignant neoplasm. Additionally, certain individuals are at risk of developing neoplasm. For example, several large epidemiology based studies have indicated that individuals with an insulin-like growth factor (IGF-I) concentration in the upper part of the normal range seem to have an increased risk of developing a number of different malignant neoplasms, including breast, colon and/or prostate cancers. Further, there are reports that acromegalic individuals have an increased risk of developing certain types of neoplasms, particularly colon cancer.

Anti-neoplastic agents, including camptothecins such as Irinotecan, are known to be used in the treatment of various neoplasms. However, there continues to be a substantial need for improved methods and compositions for treating neoplasms.

### SUMMARY OF THE INVENTION

Accordingly, it is an object of this invention to provide methods and compositions for treating, preventing and/or delaying onset of neoplasms.

These and additional objects are provided by the methods and compositions of the present invention. In one aspect, the invention is directed to methods of treating, preventing and/or delaying onset of a neoplasm. The methods comprise administering to an individual an anti-neoplastic agent and a growth hormone receptor antagonist.

In a further embodiment, the invention is directed to methods for treating, preventing and/or delaying onset of a neoplasm, which methods comprise administering to an individual Irinotecan hydrochloride and a polyethylene glycol (PEG)-modified human growth hormone, wherein the PEG-modified human growth hormone comprises from about 4 to about 5 covalently bound PEG molecules, and wherein each PEG molecule has an average molecular weight of about 5,000 Da.

In yet a further embodiment, the invention is directed to methods of reducing tumor weight or volume, which methods comprise administering to an individual an anti-neoplastic agent and a growth hormone receptor antagonist.

In yet a further embodiment, the invention is directed to compositions comprising an anti-neoplastic agent and a growth hormone receptor antagonist.

The present methods and compositions are useful for treating, preventing and/or delaying onset of neoplasms. Treatment and/or prevention of metastasis may be obtained in specific embodiments. Additional embodiments, objects and advantages of the invention will become more fully apparent in view of the following detailed description.

### BRIEF DESCRIPTION OF THE DRAWING

FIGS. 1A-1D illustrate the effects of administering vehicle alone, Pegvisomant alone, Irinotecan alone, and a combination of Pegvisomant and Irinotecan, respectively, on splenic tumor volume and the number and mass of hepatic metastases in BALB/c mice injected with the mouse colon carcinoma cell line, CT-26, as described in the Example.

### DETAILED DESCRIPTION

The present invention is directed to methods for treating, preventing and/or delaying onset of a neoplasm and methods for reducing tumor weight or volume. These methods comprise administering to an individual an anti-neoplastic agent and a growth hormone receptor antagonist. The present invention is also directed to compositions which comprise an anti-neoplastic agent and a growth hormone receptor antagonist.

As used herein, “individual” is intended to refer to an animal, including but not limited to humans, mammals or rodents, who have a neoplasm or are at risk for developing a neoplasm. Individuals at risk for developing a neoplasm include, but are not limited to, individuals with a genetic abnormality, either acquired or inherent, or individuals with a predisposition for developing a neoplasm, such as acromegalic individuals. As used herein, “treatment” is intended to refer to the reduction in the weight and/or volume of a neoplasm, while prevention and delay are intended to refer to prevention or the delay respectively in the clinical onset or manifestation of a neoplasm after the administration of an anti-neoplastic agent and a growth hormone receptor antagonist. In one embodiment of the present invention, the neoplasm is a malignant neoplasm (cancer), which includes, but is not limited to, colon cancer, breast cancer, prostate cancer or combinations thereof. In another embodiment of the present invention, the neoplasm is a benign neoplasm, which includes, but is not limited to, meningioma.

In developing the present invention, the inventors have determined that administration of a combination of an anti-neoplastic agent and a growth hormone receptor antagonist is superior to administration of an anti-neoplastic agent alone.

One skilled in the art will appreciate the various anti-neoplastic agents known to treat, prevent or delay onset of neoplasms. In one embodiment the anti-neoplastic agent comprises a camptothecin, for example, a camptochetic derivative such as Irinotecan, or a pharmaceutically acceptable salt or ester thereof. Irinotecan is disclosed in the Miyasaka et al U.S. Pat. No. 4,604,463, incorporated herein by reference, and is a commonly used agent in the treatment of neoplasms, including, but is not limited to, colon cancer. Irinotecan hydrochloride is a semisynthetic derivative of camptothecin, an alkaloid extract from plants such as Camptotheca acuminats, and is a topoisomerase-1 inhibitor. The chemical name of Irinotecan is (4S)-4,11-diethyl-4-hydroxy-9-((4-piperidinopiperidino)carbonyloxy)-1H-pyrano (3′,4′:6,7)indolizino(1,2-b)quinoline-3,14(4H,12H) dione hydrochloride. Its structural formula is as follows:

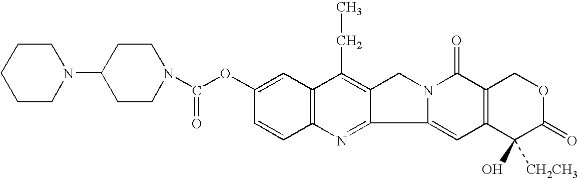

Irinotecan is available commercially, for example as a hydrochloride salt, from Pharmacia under the tradename Camptosar®.

Other anti-neoplastic agents are known in the art and are suitable for use in the present methods and compositions. For example, it is known in the art to employ various active agents in the treatment of neoplasms. Such agents are regarded within the scope of the present invention as anti-neoplastic agents and include, but are not limited to, anthracyclines, for example doxorubicin and epirubicin, taxanes, for example paclitaxel and docetaxel, epothilones, other alkylating agents such as cyclophosphamide, platinum-based agents such as cis-platin, carboplatin and oxaliplatin, fluoropyrimadines, for example 5-FU and capcitidine, antimetabolites, for example methotrexate, SERMs, aromatase inhibitors, LHRH agonists and antagonists, estrmustine, ribonucleoside reductase inhibitors, for example gemcytabine, EGFR antagonists, and antiangiogenesis agents.

The growth hormone (GH) employed in the growth hormone receptor antagonist may comprise human growth hormone, wild-type or recombinant, or variants thereof exhibiting growth hormone activity. Human growth hormone (hGH) is a 22,000 Dalton pituitary hormone known to exhibit a multitude of biological effects, including linear growth (somatogenesis), lactation, activation of macrophages, and insulin-like and diabetogenic effects, among others. Chawla, Annu. Rev. Med., 34:519 (1983); Edwards et al, Science, 239:769 (1988); Isaksson et al, Annu. Rev. Physiol., 47:483 (1985); Thomer et al, J. Clin. Invest., 82:745 (1988); Hughes et al, Annu. Rev. Physiol., 47:469 (1985). These biological effects derive from the interaction between hGH and specific cellular receptors.

hGH is a member of a family of homologous hormones that include placental lactogens, prolactins, and other genetic and species variants of growth hormone. hGH is unusual among these in that it exhibits broad species specificity and binds to either the cloned somatogenic or prolactin receptor. The cloned gene for hGH has been expressed in a secreted form in E. coli, Chang et al, Gene, 55:189 (1987), and its DNA and amino acid sequences have been reported, Goeddel et al, Nature, 281:544 (1979); Gray et al, Gene, 39:247 (1985). The receptor and antibody epitopes of hGH have been identified by homolog-scanning mutagenesis and alanine-scanning mutagenesis, Cunningham et al, Science, 243:1330 (1989); Cunningham et al, Science, 244:1081 (1989); WO 97/11178.

The hormone-receptor complex between hGH and the extracellular domain of its receptor (hGHbp) is known, Wells et al, Annu. Rev. Biophys. Biomol. Struct., 22:329 (1993). High-resolution structural and mutational analysis and structural analysis has shown that one molecule of hGH binds two receptor molecules sequentially using distinct sites on the hormone, called sites 1 and 2, Cunningham et al, Science, 244:1081 (1989); Cunningham et al, Science, 254:821 (1991); De Vos et al, Science, 255:306 (1991).

In addition to GH broad species specificity, the GH axis, which includes GH itself and the GH-stimulated peptide insulin-like growth factor I (IGF-I), are known as being capable of stimulating the growth of both normal and neoplastic tissues. Further, growth of human neoplasm, be it solid or hematological, can typically be modulated by altering the GH axis, at least in a controlled environment of a laboratory.

A number of naturally occurring and recombinant mutants or variants of hGH are known and suitable for use herein as the growth hormone receptor antagonist. See, for example, Kostyo et al, Biochem. Biophys. Acta, 925:314 (1987); Lewis et al, J. Biol. Chem., 253:2679 (1978); Tokunaga et al, Eur. J. Biochem., 153:445 (1985); WO 91/05853; WO 92/09690; WO 92/19736; WO 92/21029; and WO 97/11178. As used herein, the term “variant” encompasses both naturally occurring and recombinant derivatives of hGH in which one or more of the amino acids of hGH are substituted or deleted, provided that the derivatives exhibit hGH activity. In a specific embodiment, hGH variant B2036 as disclosed in WO 97/11178, incorporated herein by reference, is employed in the methods of the invention as disclosed herein.

The hGH variant B2036 has the following substitutions:

H18D, H21N, G12OK, R167N, K168A, D171S, K172R, E174S, I179T.

WO 97/11178 discloses that the G120K substitution is added to generate a better antagonist candidate, although other substitutions at that position are acceptable. Any amino acid can be substituted at G120 to generate an antagonist; more preferably the substitution is lysine, arginine, tryptophan, tyrogine, phenylalanine, or glutamate. The K168A and the K172R substitutions are added to reduce the number of sites available for pegylation at the hormone-receptor site I binding interface. In contrast, the G120K substitution makes available an additional lysine for pegylation while providing an effective site 2 block. It is expected that B2036 could be further modified by restoring the glycine at residue 120, thereby generating a candidate for use as an agonist that is expected to have reduced antigenicity in humans in comparison with other variants, for example 852d.

The vector used for expression of the B2036 variant in E. coil is pMY233. Plasmid pMY223 is based on the well-characterized plasmid pBR322 and is similar to the hGH production plasmid pHGH4R (Chang et al, Gene, 55:189 (1987)), except that the B2036 coding sequence replaces the hGH coding sequence. pMY223 encodes resistance to tetracycline antibiotics, but unlike pBR322 is sensitive to B-lactam antibiotics (penicillin, ampicillin, etc.).

The amino acid differences between the B2036 variant encoded by pMY223 and the wild-type human growth hormone sequence are shown below, along with the codons at these sites.

| Wild-type | Amino | B2036 | B2036 |
|---|---|---|---|
| amino acid | acid # | amino acid | codon |
| His | 18 | Asp | GAC |
| His | 21 | Asn | AAC |
| Gly | 120 | Lys | AAG |
| Arg | 167 | Asn | AAC |
| Lys | 168 | Ala | GCG |
| Asp | 171 | Ser | AGC |
| Lys | 172 | Arg | AGG |
| Glu | 174 | Ser | AGC |
| Ile | 179 | Thr | ACC |

Further details of the B2036 variant, including embodiments for production, are disclosed in WO 97/11178, incorporated herein by reference.

In one embodiment, the growth hormone receptor antagonist is a polyethylene glycol (PEG)-modified growth hormone. As used herein, the terms “polyethylene glycol modified”, “PEG-modified” and “pegylated” are used synonymously to refer to a growth hormone having one or more polyethylene glycol groups covalently bound thereto.

The growth hormone receptor antagonist may be covalently attached, or conjugated, to one or more polyethylene glycol groups. Such conjugation produces a growth hormone conjugate having a greater actual molecular weight than the unmodified growth hormone. As used herein, the term “actual molecular weight” refers to the molecular weight, as measured, for example, by mass spectrometry (e.g., matrix-assisted laser desorption ionization mass spectrometry). For example, the actual molecular weight of the hGH variant conjugates is usually at least about 30 kD, more specifically, in the range of about 35 kD to about 55 kD, and even more specifically, in the range of about 40 kD to about 50 kD. Generally, the actual molecular weight of the hGH variant conjugate does not exceed 100 kD.

The polyethylene glycol can be conjugated to the growth hormone molecule at one or more amino acid residues, including lysine residues. One or more additional water-soluble poly(alkylene oxide) polymers having a linear or branched chain may be employed in addition to polyethylene glycol, but polyethylene glycol alone is preferred. The average molecular weight of the PEG can range from about 500 to about 30,000 Daltons (Da), more specifically, from about 1,000 to about 25,000 Da and even more specifically, from about 4,000 to about 20,000 Da. In one embodiment, pegylation is carried out with PEG having an average molecular weight of about 5,000 Da (hereinafter “PEG(5000)”). In another embodiment, pegylation is carried out with PEG having an average molecular weight of about 20,000 Da.

In one embodiment, the reaction conditions are adjusted to maximize production of growth hormone conjugated to from one to about six molecules of polyethylene glycol, while in another, more specific embodiment, the reaction conditions are adjusted to maximize production of growth hormone conjugated to from about four to about five molecules of polyethylene glycol. In yet another embodiment, the reaction conditions are adjusted to maximize production of growth hormone molecules conjugated to one molecule of PEG. In a variation of these embodiments, a branched-chain PEG may be employed.

PEG preparations that are commercially available, and suitable for use in the present invention, are nonhomogeneous preparations that are sold according to average molecular weight. For example, PEG(5000) preparations typically contain molecules that vary slightly in molecular weight, usually ±500 Da.

A variety of methods for pegylating proteins have been described. See, e.g., U.S. Pat. No. 4,179,337 to Davis et al, disclosing the conjugation of a number of hormones and enzymes to PEG and polypropylene glycol to produce physiologically active non-immunogenic compositions. Generally, a PEG having at least one terminal hydroxy group is reacted with a coupling agent to form an activated PEG having a terminal reactive group. This reactive group can then react with the α- and ε-amines of proteins to form a covalent bond. Conveniently, the other end of the PEG molecule can be “blocked” with a non-reactive chemical group, such as a methoxy group, to reduce the formation of PEG-crosslinked complexes of protein molecules.

For pegylation of hGH or an hGH variant, the activated PEG is one that can react with the variant under conditions that do not destroy Site 1 binding activity. For agonist hGH variants, Site 2 binding activity must also be preserved. Furthermore, for agonist and antagonist hGH variants, activated PEGs that introduce a toxic linking group into the conjugate are usually avoided. Suitable activated PEGs can be produced by a number of conventional reactions. For example, an N-hydroxysuccinimide ester of a PEG (M-NHS-PEG) can be prepared from PEG-monomethyl ether (which is commercially available from Union Carbide) by reaction with N,N′-dicyclohexylcarbodiimide (DCC) and N-hydroxysuccinimide (NHS), according to the method of Buckmann et al, Makromol. Chem., 182:1379 (1981). In addition, a PEG terminal hydroxy group can be converted to an amino group, for example, by reaction with thionyl bromide to form PEG-Br, followed by aminolysis with excess ammonia to form PEG-NH2. The PEG-NH2 is then conjugated to the growth hormone of interest using standard coupling reagents, such as Woodward's Reagent K. Furthermore, a PEG terminal —CH₂OH group can be converted to an aldehyde group, for example, by oxidation with MnO₂. The aldehyde group is conjugated to the protein by reductive alkylation with a reagent such as cyanoborohydride.

Alternatively, activated PEGs suitable for use in the present invention can be purchased from a number of vendors. For example, Shearwater Polymers, Inc. (Huntsville, Ala.) sells M-NHS-PEG as “SCM-PEG” in addition to a succinimidyl carbonate of methoxy-PEG (“SC-PEG”) and methoxy-PEG succinimidyl propionate (“SPA-PEG”; hereinafter referred to as “M-SPA-PEG” to indicate the presence of the methoxy blocking group). Shearwater Polymers also sells a branched-chain PEG having two 10,000 D chains (hereinafter “NHS-PEG2(20,000)”).

The degree of pegylation of a growth hormone for use in the present invention can be adjusted to provide a desirably increased circulatory half-life, increased physicochemical stability, and/or increased bioavailability of the growth hormone upon delivery via, for example, the pulmonary route, particularly as compared to the corresponding non-pegylated growth hormone.

The sites of pegylation of a growth hormone are also somewhat constrained by the reactivities of the various primary amines. For example, a potential lysine in the Site 1 hormone-receptor binding interface of the B2036 variant (K41) is relatively unreactive with N-SPA-PEG(5000). Thus, moderately pegylated B2036 variant preparations, having on the order of four to six PEGs per variant molecule, retain the ability to bind hGH receptor at Site 1, despite the presence of a potential pegylation site at this binding interface.

Standard mutagenesis techniques can be used to alter the number of lysines in the growth hormone. Thus, to the extent that amino acid substitutions introduce or replace lysines, hGH variants of the present invention can contain a greater or lesser number of potential pegylation sites than wild-type hGH. The B2036 variant contains nine potential pegylation sites, one fewer than wild-type hGH. Furthermore, amino acid substitutions introducing or replacing lysines alter the locations of potential pegylation sites. For example, in the B2036 variant, the K168A and the K172R substitutions reduce the number of sites available for pegylation at the hormone-receptor Site 1 binding interface. The replacement of G120 with a different amino acid disrupts hGH binding at Site 2, converting the molecule to an hGH antagonist. The substitution of lysine for glycine at this position provides an additional potential pegylation site in Site 2, which is expected to impair any residual binding at this site. The degree and sites of pegylation can also be manipulated by adjusting reaction conditions, such as the relative concentrations of the activated PEG and the protein as well as the pH. Suitable conditions for a desired degree of pegylation can be determined empirically.

Pegylation of hGH variants, such as B2036, may be carried out by any convenient method. A commercially available product of this type comprises Pegvisomant. In an exemplary embodiment, hGH variants are pegylated with H-SPA-PEG(5000). Briefly, solid SPA-PEG(5000) is added, with stirring, to an aqueous solution of hGH variant at room temperature. Typically, the aqueous solution is buffered with a buffer having a pK, near the pH at which the reaction is to be carried out (generally about pH 4-10). Examples of suitable buffers for pegylation at pH 7.5, for instance, include HEPES, phosphate, borate, Tris-HCl, EPPS, and TES. The pH is continuously monitored and adjusted if necessary. The reaction is allowed to continue for about one to about two hours. The reaction products are then subjected to hydrophobic interaction chromatography to separate pegylated hGH variants from free H-SPA-PEG(5000) and any high-molecular weight complexes of the pegylated hGH variant. High-molecular weight complexes arise when unblocked PEG is activated at both ends of the molecule, crosslinking hGH variant molecules. The conditions during hydrophobic interaction chromatography are such that free M-SPA-PEG(5000) flows through the column, while any crosslinked pegylated hGH variant complexes elute after the desired forms, which contain one hGH variant molecule conjugated to one or more PEG groups. Suitable conditions vary depending on the relative sizes of the crosslinked complexes versus the desired conjugates and are readily determined by those skilled in the art. The eluent containing the desired conjugates is concentrated by ultrafiltration and desalted by diafiltration.

This preparation represents a heterogeneous mixture of PEG-hGH variant conjugates having between three and six PEG groups per molecule of hGH variant. In one embodiment, this mixture may be subjected to an additional purification step that produces a more homogeneous preparation of pegylated hGH variants. More specifically, the mixture is subjected to cation exchange chromatography to fractionate the pegylated hGH variants according to the extent of pegylation.

The conditions are such that the more highly pegylated hGH variants having a greater number of PEG groups elute early in the gradient. In this manner, it is possible to obtain a pool of pegylated hGH variants containing primarily one or two forms. As used hereinafter, a “form” of a pegylated hGH variant is an PEG-hGH variant conjugate containing a particular number of PEG groups. Accordingly, different “forms” of a pegylated hGH variant have different numbers of PEG groups conjugated to the same hGH variant. In an exemplary embodiment, a pool of pegylated hGH variants is obtained that contains primarily two forms, namely, conjugates having 4 or 5 PEGS per molecule of hGH variant (hereinafter a “PEG-4/5hGH variant preparation”). This pool can then be concentrated, desalted, and formulated for administration.

A composition containing a pegylated hGH variant for use in the present invention may be heterogeneous, i.e., containing several or many PEG-hGH forms, or homogeneous, i.e., containing a single PEG-hGH form. Typically, the composition contains at least 70% of one or two forms of PEG-hGH variant conjugates preferably, at least 80% of one or two forms; and more preferably, at least 90% of one or two forms.

While a pegylated form of growth hormone receptor antagonist is suitable for use in the invention, it is equally within the scope of the present methods and compositions to employ a non-pegylated form of growth hormone receptor antagonist.

One skilled in the art will recognize the various methods that may be employed for the administration of an anti-neoplastic agent and a growth hormone receptor antagonist. In one embodiment of the invention, the anti-neoplastic agent is administered parenterally, locally or systemically. In a further embodiment of the invention, the growth hormone receptor antagonist is administered by pulmonary delivery, parenterally, locally or systemically, or combinations thereof. Examples of parenteral administration include, but are not limited to, subcutaneous, intramuscular, intravenous, intraarterial, and intraperitoneal. The parenteral administration may be by continuous infusion (using, e.g., minipumps such as osmotic pumps), or by injection using, e.g., intravenous or subcutaneous means. In one embodiment, the growth hormone receptor antagonist and/or the anti-neoplastic agent are administered subcutaneously. The administration can also be as a single bolus or by slow-release depot formulation. The growth hormone receptor antagonist may also be delivered via the pulmonary route in either liquid or solid form. The pegylated form of growth hormone receptor agonist is suitable for use in this embodiment. In a specific embodiment, pegylated growth hormone is delivered in the form of a fine liquid aerosol in which drops or particles have a size of from about 1 to about 3 microns in diameter. The liquid aerosol is formed from a liquid formulation of the growth hormone receptor antagonist, which, in turn, may be formed by reconstitution of a lyophilized powder. Moreover, one skilled in the art will recognize that the growth hormone receptor antagonist and the anti-neoplastic agent may be administered together or separately.

The anti-neoplastic agent and the growth hormone receptor antagonist are administered to an individual in a therapeutically effective amount. One skilled in the art will appreciate that the dosage and administrative regimen of these compounds depends on several factors such as the potency of the anti-neoplastic agent, the mode of administration, the age and weight of the patient, the severity of the condition to be treated, and the like. This is considered to be within the skill of the artisan, and one can review the existing literature on the components to determine optimal dosing.

As used herein, “pharmaceutically acceptable” refers to those properties and/or substances that are acceptable to the patient from a pharmacological/toxicological point of view and to the manufacturing pharmaceutical chemist from a physical/chemical point of view regarding composition, formulation, stability, patient acceptance and bioavailability.

Anti-neoplastic agents are often dosed by square meter (m²) of body surface area (BSA). BSA is often chosen rather than body weight as the basis for calculation for two reasons. First, BSA has been demonstrated to provide a more accurate cross-species comparison of activity and toxicity for certain drugs. Second, BSA can be more closely correlated with cardiac output, which determines the blood flow to the liver and kidneys, thus influencing drug elimination. While not intending to be limiting, exemplary average values of BSA are about as follows: man: 1.9 m²; woman: 1.6 m²; 9 year old child: 1.07 m²; 10 year old child: 1.14 m²; and 12-13 year old child: 1.33 m². One skilled in the art will appreciate that these are only general guidelines and variations thereof may be determined using techniques well within the ability of those skilled in the art. Dosages may range, for example, from about 50 to about 500 mg/m² per administration and design of administration regimes is within the ordinary skill in the art.

As a more specific example, Irinotecan may be administered in a regimen wherein a dose of 125 mg/m² of Irinotecan is given intravenously over a 90 minute period once weekly for 4 weeks, followed by a 2-week rest period. Thereafter, additional courses of treatment may be repeated every 6 weeks (4 weeks on therapy, followed by 2 weeks off therapy). Subsequent doses may be adjusted to as high as 150 mg/m² or to as low as 50 mg/m² in 25-mg/m² to 50-mg/m² increments depending upon individual patient tolerance of treatment, as judged by the attending physician; or a regimen wherein a dose of 350 mg/m² of irinotecan is given intravenously over 90 minutes, every third week, may be suitable.

As a general proposition, the total pharmaceutically acceptable amount of the growth hormone receptor antagonist administered parenterally per dose is in the range of about 1 μg/kg/day to about 100 mg/kg/day of patient body weight, although, as noted above, this is subject to therapeutic discretion. Usually, this dose is between about 0.01 and about 10 mg/kg/day, and more usually for humans between about 0.01 and about 1 mg/kg/day. If given continuously, the growth hormone receptor antagonist is typically administered at a dose rate of about 1 μg/kg/hour to about 50 μg/kg/hour, either by one to four injections per day or by continuous subcutaneous infusions, for example, using a mini-pump. An intravenous bag solution may also be employed.

### EXAMPLE

This example demonstrates that the administration of the growth hormone receptor antagonist, Pegvisomant, in combination with administration of the anti-neoplastic agent, Irinotecan, provides significant improvement over the therapeutic effect of Irinotecan when administered alone. The effects of Pegvisomant and Irinotecan, alone and in combination, are assessed on CT-26 cells (mouse colon carcinoma cell line) injected into the spleen of BALB/c mice. This model allows for assessment of treatment efficacy on the primary tumor (spleen) and on hepatic metastases.

Growth Hormone Receptor Antagonist

Pegvisomant comprises a PEG-modified 191 amino acid residue analog of common human growth hormone, specifically B2036, comprising an average of from about 5 to about 5 polyethylene glycol molecules, with the average molecular weight of each polyethylene glycol molecule being about 5,000 Da. The B2036 protein is designed to act as an antagonist of the hGH receptor by the exchange of an amino acid residue in the hGH backbone as described above. Since the PEG related B2036 protein is of the same number of amino acid residues as hGH, this PEG related protein may be viewed as a model variant of a PEG-related hGH.

Cell Culture/Animals

CT-26 cells (mouse colon carcinoma) are grown in DMEM with 10% fetal bovine serum until the cells are approximately 50% confluent. Trypsin is used to lift adherent cells from the surface of tissue culture flask. The cells are then washed and suspended in Hank's buffered saline solution. BALB/c mice (Jackson Laboratories, Bar Harbor, Me.), 5-8 weeks of age, are used in this example. Initially, the spleen is mobilized using a left flank incision. Direct splenic injections of CT-26 cells is performed (1×10⁴ viable cells, 0.05 ml total volume). Wound clips are used to close the incision. This is a syngenic colon cancer model so the host animals are not immunocompromised.

Treatment

Mice are either treated with vehicle (saline) alone, subcutaneous injections of Pegvisomant (3.33, 5.0, or 10.0 mg) once daily, Irinotecan (100 mg/kg on post-inoculation days 7, 14, 21) and saline, or a combination of the Irinotecan and Pegvisomant. In a further experiment, octreotide injections, 2 μg twice a day, are administered subcutaneously. Injections are administered on the flank opposite the side of the wound clips from the splenic injection using a 25-gauge needle on a tuberculin syringe.

Tumor Harvesting

Mice are sacrificed on day 24. Measurements are made of the primary tumor (spleen), metastatic tumor burden (liver weight), and mean number of liver metastases. Treatment groups are compared using a standard t-test. P<0.05 compared to vehicle; P<0.05 compared to vehicle and Irinotecan.

Serum IGF-I Assay

Mouse serum IGF-1 is measured by radioimmunoassay (RIA) after acid-ethanol extraction. Briefly, the serum/extraction mixture is incubated at room temperature for 2 hours, centrifuged, and the supernatant is diluted 1:200 for analysis. IGF-I concentrations are the measured by RIA using a polyclonal rabbit antibody (Nicohls Institute, San Juan Capistrano, Calif.). The assay standards are created with recombinant human IGF-I (Amersham International, Bucks, UK).

RNA-PCR

Semi-quantitive Polymerase Chain Reaction (PCR) is performed to determine the relative amounts of IGF-I and IGF-II messenger RNA in the harvested tumor specimens. For each reaction, 1 μg of total RNA is used. Reverse transcription is performed. The downstream primer for each of the subsequently described PCR primer pairs is employed as a gene-specific primer for the reverse transcription.

| | |
|---|---|
| IGF-I | |
| upstream | 5′-ATGGGAAAAATCAGCAGTCTTCC-3′ |
| downstream | 5′-CTGGGTCTTGGGCATGTCGGTGTG-3′ |
| | PCR product 401. |
| IGF-II | |
| upstream | 5′-GGAGACAGTCCGCGGGACG-3′ |
| downstream | 5′-GATGGTACTACATTGCAG-3′ |
| | PCR product size 227. |
| β-actin | |
| upstream | 5′-GACAGGATGCAGAAGGAG-3′ |
| downstream | 5′-CTAGAAGCATTTGCGGTG-3′ |
| | PCR product size 200. |

PCR is performed (25 cycles) with an end labeled primer and products are separated from unincorporated radiolabeled primer by electrophoresis through an acrylamide gel. The amount of target message in each individual sample is quantified using a phosphorimager. To correct for loading differences, sample values are normalized for β-actin message content.

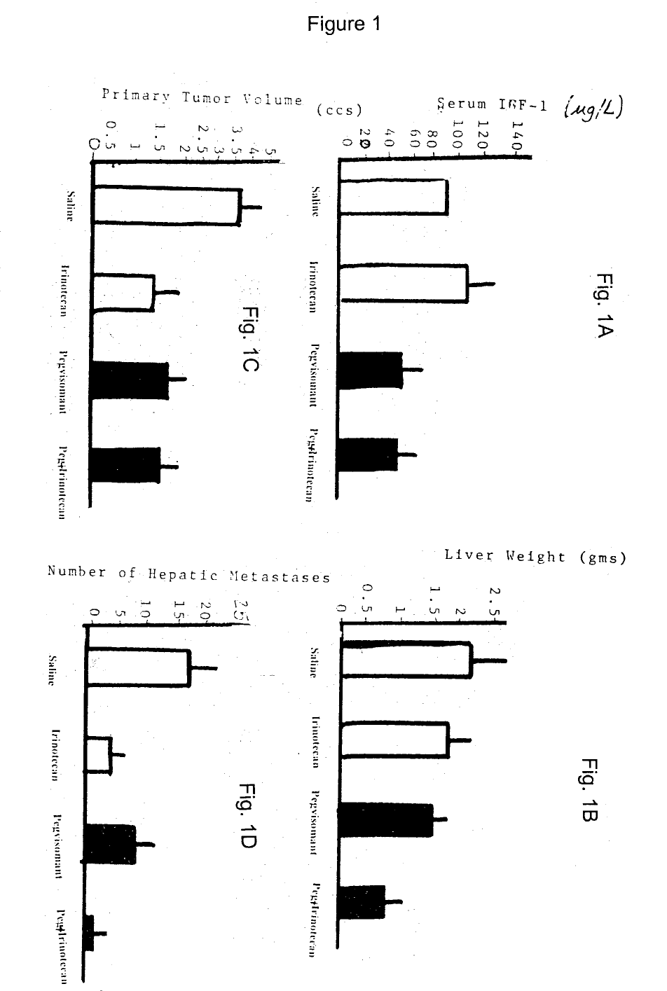

FIGS. 1A-1D illustrate the treatment efficacy on the primary tumor in the spleen and on the number and mass of hepatic metastases of the four treatment groups, namely saline, Pegvisomant (5.0 mg/day), Irinotecan (100 mg/kg on post-inoculation days 7, 14, 21), and Pegvisomant+Irinotecan. Specifically, FIG. 1A illustrates the Serum IGF-1 concentration (μg/L); FIG. 1B illustrates the liver weight (grams); FIG. 1C illustrates the primary tumor volume (ccs); and FIG. 1D illustrates the number of hepatic metastases of the four treatment groups. Combination therapy with Pegvisomant and Irinotecan is superior to treatment with Irinotecan alone for all parameters measured in the experiment with CT-26 colon cancer cells. Beneficial effects of Pegvisomant in combination with Irinotecan are seen on both the primary tumor and on hepatic metastases. Pegvisomant therapy potentiates Irinotecan action, particularly with regard to inhibiting the development of metastatic disease in the liver.

The mean values±standard errors of the serum collected when the animals are sacrificed and assayed are for IGF-I concentrations (μ/L) as follows: (Serum) 87.6±9.1, (Pegvisomant) 50±5.3, (Irinotecan) 128.6±5.9, (Pegvisomant+Irinotecan) 53±2.9. Baseline values in mice with no tumor burden is approximately 200 μ/L. The low value in the saline group is probably reflective of the animals significantly advanced illness in the days prior to sacrifice.

Of the 10 animals treated with the combination of Pegvisomant and Irinotecan, only 4 developed hepatic metastases. This is only 50% of the number of animals that developed metastases with placebo or Irinotecan therapy. Given that metastatic disease to the liver is one of the most difficult complications to manage and a significant contributor to colon cancer mortality, the ability of Pegvisomant/Irinotecan combination therapy to reduce such metastases is of marked clinical significance.

This example demonstrates that the administration of a growth hormone receptor antagonist, such as Pegvisomant, in conjunction with an anti-neoplastic agent, such as Irinotecan, has a significant anti-tumor effect on animal models of three tumor types (colon cancer, breast cancer and meningiomas) that are known to be responsive to GH and/or IGF-I.

The foregoing description of the various embodiments in the invention has been presented for the purposes of illustration description. It is not intended to be exhaustive or to limit the invention to the precise form disclosed. Many alternatives, modifications and variations will be apparent to those skilled in the art of the above teaching. Accordingly, this invention is intended to embrace all alternatives, modifications and variations that have been discussed herein and others that fall within the spirit and broad scope of the claims.

What is claimed is:

1. A method of treating, preventing or delaying clinical onset of a neoplasm, comprising administering to an individual an anti-neoplastic agent and a growth hormone receptor antagonist.

2. The method according to claim 1, wherein the neoplasm is a malignant neoplasm.

3. The method according to claim 2, wherein the malignant neoplasm comprises colon cancer, breast cancer, prostate cancer, or combinations thereof.

4. The method according to claim 1, wherein the neoplasm is a benign neoplasm.

5. The method according to claim 4, wherein the benign neoplasm is a meningioma.

6. The method according to claim 1, wherein the anti-neoplastic agent comprises a camptothecin, or a pharmaceutically acceptable salt or ester thereof.

7. The method according to claim 1, wherein the anti-neoplastic agent comprises an Irinotecan, or a pharmaceutically acceptable salt or ester thereof.

8. The method according to claim 1, wherein the growth hormone receptor antagonist comprises human growth hormone.

9. The method according to claim 1, wherein the growth hormone receptor antagonist comprises a recombinant human growth hormone or a variant of recombinant human growth hormone.

10. The method according to claim 9, wherein the growth hormone receptor antagonist comprises B2036.

11. The method according to claim 1, wherein the growth hormone receptor antagonist comprises a PEG-modified growth hormone.

12. The method according to claim 1, comprising administering to an individual Irinotecan hydrochloride, or a pharmaceutically acceptable salt or ester thereof, and a polyethylene glycol (PEG)-modified human growth hormone, wherein the PEG-modified human growth hormone comprises from about four to about five covalently bound PEG molecules, and wherein each PEG molecule has an average molecular weight of about 5,000 Da.

13. A composition comprising an anti-neoplastic agent and a growth hormone receptor antagonist.

14. The composition according to claim 13, wherein the anti-neoplastic agent comprises a camptothecin, or a pharmaceutically acceptable salt or ester thereof.

15. The composition according to claim 13, wherein the anti-neoplastic agent comprises Irinotecan, or a pharmaceutically acceptable salt or ester thereof.

16. The composition according to claim 13, wherein the growth hormone receptor antagonist comprises human growth hormone.

17. The composition according to claim 13, wherein the growth hormone receptor antagonist comprises a recombinant human growth hormone or a recombinant human growth hormone variant.

18. The composition according to claim 17, wherein the growth hormone receptor antagonist comprises B2036.

19. The composition according to claim 13, wherein the growth hormone receptor antagonist comprises a PEG-modified growth hormone.

20. A method of reducing tumor weight or volume, comprising administering to an individual an anti-neoplastic agent and a growth hormone receptor antagonist.

21. The method according to claim 20, wherein the tumor is a malignant neoplasm.

22. The method according to claim 21, wherein the malignant neoplasm comprises colon cancer, breast cancer, prostate cancer, or combinations thereof.

23. The method according to claim 20, wherein the tumor is a benign neoplasm.

24. The method according to claim 23, wherein the benign neoplasm is meningioma.

25. The method according to claim 20, wherein the anti-neoplastic agent comprises a camptothecin, or a pharmaceutically acceptable salt or ester thereof.

26. The method according to claim 20, wherein the anti-neoplastic agent comprises Irinotecan, or a pharmaceutically acceptable salt or ester thereof.

27. The method according to claim 20, wherein the growth hormone receptor antagonist comprises human growth hormone.

28. The method according to claim 20, wherein the growth hormone receptor antagonist comprises a recombinant human growth hormone or a variant of recombinant human growth hormone.

29. The method according to claim 28, wherein the growth hormone receptor antagonist comprises B2036.

30. The method according to claim 20, wherein the growth hormone receptor antagonist comprises a PEG-modified growth hormone.
